(19) United States
(12) Patent Application Publication
(10) Pub. No.: US 2007/0215162 A1
(43) Pub. Date: Sep. 20, 2007

(54) Visualization airway apparatus and methods for selective lung ventilation

(75) Inventors: Raymond Glassenberg, Wilmette, IL (US); Zebadiah Kimmel, Brookline, MA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: EZC Medical LLC, San Francisco, CA (US)

(21) Appl. No.: 11/710,007

(22) Filed: Feb. 23, 2007

Related U.S. Application Data
(63) Continuation-in-part of application No. 11/303,343, filed on Dec. 16, 2005.

(51) Int. Cl. A62B 9/06; A61M 16/00

(52) U.S. Cl. 128/207.15; 128/207.14

(57) ABSTRACT

An airway device and method of use are provided, in which the airway device includes a dual lumen airway suitable for use as an endobronchial tube and having a sensor and a sensor monitoring device. In an embodiment of the device, the airway device includes imaging apparatus, optionally textured balloons, and image display device for monitoring the position of the device, thereby facilitating placement of the device and monitoring ability to determine a change in positioning.

### CROSS-REFERENCE TO RELATED APPLICATIONS

This application is a continuation-in-part of patent application Ser. No. 11/303,343, filed Dec. 16, 2005.

### FIELD OF THE INVENTION

The present invention relates to airway apparatus equipped with visualization capabilities and capable of providing selective ventilation to either of the lungs.

### BACKGROUND OF THE INVENTION

In the medical profession, a patient may require surgery to treat a traumatic injury or a medical condition. If this surgery is to the heart, lungs, or other thoracic organs, a surgeon or other caregiver may have to access the organ by making an incision in the chest, known as a thoracotomy.

When performing a thoracotomy, a patient is typically anesthetized and an airway device is inserted into the patient's trachea to allow for mechanical ventilation or other form of delivering oxygenating gasses to the patient's lungs. Under certain circumstances, such as for a lung surgery, the airway device is a dual lumen airway device comprising a tube that terminates in one of the patient's two bronchi that branch out from the trachea. These dual lumen airway devices may be referred to as double-lumen endobronchial tubes.

A double-lumen endobronchial tube typically has two balloons located along the distal portion of the shaft, which will be referred to as the distal balloon and the proximal balloon. These devices also have two lumens, where the distal end of the first lumen is distal to the distal balloon, and the distal end of the second lumen is between the distal balloon and the proximal balloon.

When positioned within a patient, the proximal balloon is expanded in the patient's trachea, thereby limiting the ventilation pathway through the trachea to that which passes through the endobronchial tube. The distal balloon is expanded in one of the two bronchi at or near the level of the carina. Ventilation may then be selectively conducted between each of the lungs, as discussed by example below.

Assume, for example, that the distal balloon of a double-lumen endobronchial tube is expanded in a patient's left bronchus. Then, when ventilation is conducted through the first lumen, the ventilation pathway of oxygen and other gasses is directed solely to the left lung. Conversely, when ventilation is conducted through the second lumen, the distal balloon prevents the oxygen and other gasses from passing into the left lung, and these gasses are therefore directed into the right lung. In this manner, selective ventilation may be conducted for each lung.

Double-lumen endobronchial tubes are known in the art, but are also associated with known problems. For example, in a left thoracotomy, the patient may have to be turned from a supine position to a right lateral position. Frequently, the double-lumen endobronchial tube may become dislodged during the turning. As a consequence, the medical professionals then may have to pass a pediatric fiberoptic device down the second lumen to ascertain the positioning of the distal balloon.

Due to the geometric limitations imposed by the size of the lumens and that of the fiberoptic device, it is not practical to maintain fiberoptic device within the endobronchial tube during the surgical procedure. Nevertheless, it remains important to have a mechanism by which the positioning of the distal balloon may be monitored.

Given the disadvantages of the known art, it is desirable to provide an airway device and method that is capable of selective ventilation to either of a patient's lungs.

It is further desirable to provide an airway device that can allow the operator to determine the placement of the airway device without the need to use a pediatric fiberscope or otherwise unduly obstruct the ventilation pathway.

It is yet further desirable to provide an airway device that allows the operator to monitor the position of the airway device as it is being used.

### SUMMARY OF THE INVENTION

In view of the above-listed disadvantages with known devices, it is an object of the present invention to provide an airway device and method that is capable of selective ventilation to either of a patient's lungs.

It is another object of the present invention to provide an airway device that can allow the operator to determine the placement of the airway device without the need to use a pediatric fiberscope or otherwise unduly obstruct the ventilation pathway.

It is a further object of the present invention to provide an airway device that allows the operator to monitor the position of the airway device as it is being used.

These and other advantages can be accomplished by providing an airway device having two lumens and a visualization device for allowing internal visualization of the placement of the airway and ongoing monitoring of the positioning of the device.

The airway device of the present invention comprises two balloons and two lumens allowing ventilation either between the balloons or through the distal end of the device (furthest from the user). An embodiment of the airway device further comprises a visualization device mounted along a distal portion of the device such that it gathers images of nearby anatomical features. The visualization device preferably is a digital imaging device, such as a CMOS (complementary metal oxide semiconductor) or CCD (charge coupled device) chip.

Illumination devices may also be incorporated into the airway to assist the visualization device. Examples of illumination devices include, without limitation, light emitting diodes (LEDs) and infrared lights.

Some dual lumen airway devices include two lumens that terminate in a common distal end. For a double-lumen endobronchial tube having two balloons, one lumen is open at the distal end, whereas the other lumen may terminate in an opening between the two balloons. Accordingly, there may be space along the outer portion of the device between the distal end of the latter lumen and the distalmost balloon. In an embodiment of the present invention, that space is utilized as one possible location to position visualization and/or illumination components.

The visualization device may transmit signals through a wire or using wireless technology. Signals are received by an imaging device, such as a monitor, where the image may be observed by the operator or other individual.

Observation of the imaging device may allow the user to determine whether the distalmost balloon becomes dislodged during the procedure through ongoing monitoring. These changes may indicate that the airway device is no longer properly positioned, thereby allowing the user to reposition the device before the patient suffers consequential harm. Additionally, the imaging device may be used during the initial positioning of the airway device to confirm proper placement as the airway device is inserted into the patient.

In accordance with one aspect of the present invention, the dual lumen airway device is disposable and discarded after a single-use. The visualization device includes electrical lead wires that terminate in a connector that may be coupled to a reusable unit that processes the signals from the visualization device to generate images. Preferably, the airway device may be coupled to a reusable module that houses components for powering the visualization device, processing the signals generated by the visualization device, and optionally, powering the illumination device. The reusable module also may include a screen for displaying the images generated by the visualization device, or may generate an output suitable for display on a conventional display.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a side view of an embodiment of an airway device incorporating features of the invention;

FIG. 2 is a cross-sectional view of the embodiment of an airway device taken along line 2-2 shown in FIG. 1;

FIG. 3 is a side view of an embodiment of an airway device incorporating features of the invention;

FIGS. 4A-C depict steps in a method of using the embodiment of the present invention depicted in FIG. 3;

FIG. 5 depicts a side view of an embodiment of an airway device incorporating features of the invention;

FIG. 6 is a cross-sectional view of the embodiment of an airway device taken along line 6-6 shown in FIG. 5;

FIG. 7 is a schematic view of an embodiment of an airway device configured to obstruct a patient's left bronchus; and

FIG. 8 is a schematic view of an embodiment of an airway device configured to obstruct a patient's right bronchus.

### DETAILED DESCRIPTION OF THE INVENTION

The present invention is directed at a dual lumen airway device that comprises a visualization device that can assist in determining the placement of the device and identifying any subsequent repositioning. Accordingly, the user can ascertain the positioning of the device and continually monitor for inadvertent repositioning. The ability of the user to continually monitor the airway's position reduces the risk of an inadvertent repositioning remaining unnoticed.

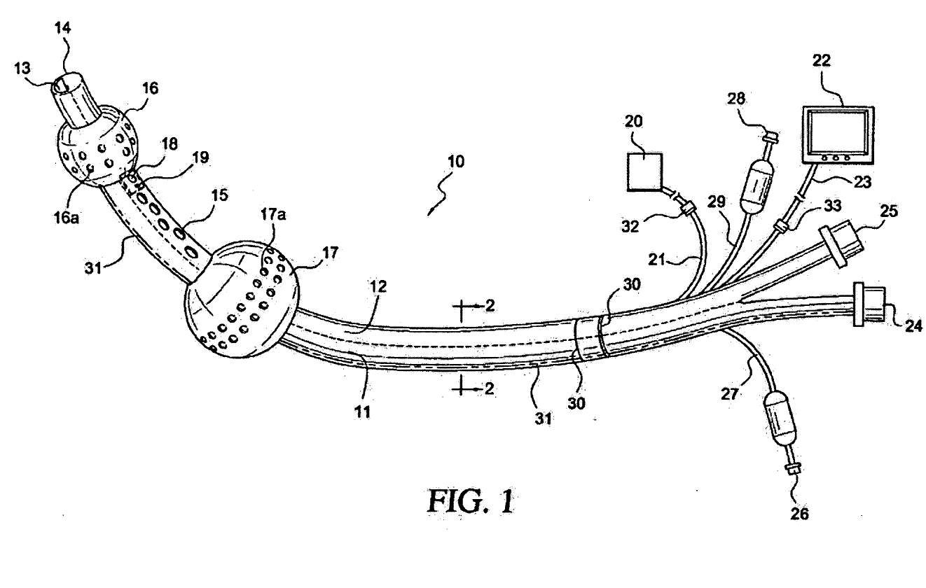

FIG. 1 depicts a preferred embodiment of the present invention that is appropriate for use in the upper airway and may be placed either in the patient's trachea or esophagus. Device 10 has tracheal lumen 11 and esophageal lumen 12. Aperture 13 of tracheal lumen 11 is located at distal end 14 of device 10. Apertures 15 of esophageal lumen 12 are located between distal balloon 16 and proximal balloon 17.

In this embodiment, balloons 16 and 17 comprise texture 16a and 17a. Texture 16a and 17a preferably comprises dimples or indentations, but may also comprise other geometries such as annular channels. Texture 16a and 17a may enhance the interaction between a bodily lumen and balloons 16 and 17. In particular, when balloons 16 and 17 are inflated, the exterior of balloons 16 and 17 will be in contact with the interior of a bodily lumen. Texture 16a and 17a may then be associated with areas of localized suction or increased contact between the interior of the bodily lumen and balloons 16 and 17.

Device 10 further comprises visualization device 18 located at least partially between distal balloon 16 and proximal balloon 17. In a preferred embodiment, visualization device 18 comprises a CMOS chip, and more preferably comprises a CMOS chip with analog output that can directly interfaced with video hardware using NTSC/PAL format. CMOS chips with analog output that can be directly interface with video hardware using NTSC/PAL format are commercially available, such as models OV7940 and OV7941 available through OmniVision Technologies, Inc., of Sunnyvale, Calif. Of course, digital outputs and other output formats are acceptable as well, and are intended to fall within the scope of the present disclosure.

Visualization device 18 is preferably configured to reduce the delivery profile of device 10. In particular, visualization device 18 may be configured with a pixel array or other image gathering component remote from the supporting circuitry. By configuring visualization device 18 as described, the circuitry may be positioned in esophageal lumen 12 distal of apertures 15 in space that may otherwise remain unused, as described in greater detail below. The circuitry may be disposed on a circuit board, which may be rigid or flexible or otherwise, as is known in the art.

In a preferred embodiment, visualization device 18 provides analog output readable by hardware using NTSC/PAL technology. Hence, the absence of an analog-to-digital converter reduces number of required components incorporated into visualization device 18. Visualization device 18 further may be reduced in size by omitting any infrared filter that would otherwise be commonly associated with a CMOS chip.

In an alternative embodiment, visualization device 18 may comprise a commercially available CMOS chip, such as a ⅓ inch CMOS chip or smaller, as is per se known in the art. The imaging portion of visualization device 18 preferably is embedded or potted in the wall of esophageal lumen 12 and is separated from the outside environment by an optically clear window or lens.

As balloons 16 and 17 are inflated, device 10 typically becomes aligned near the centerline of the trachea or esophagus. As a result, visualization device 18 will be positioned at a distance from the interior wall of the bodily orifice that is geometrically related to the diameter of balloons 16 and 17. As such, visualization device 18 may be selected such that it has a preset focal length appropriate for the distance that it will be offset from the interior wall of the bodily lumen. Alternatively, visualization device 18 may have a variable focal length, such as one that is adjustable by the user.

Illumination device 19 is located in proximity to visualization device 18, such that illumination device 19 provides visible light, infrared light, or other illumination appropriate for visualization device 18. In the embodiment shown, illumination device 19 comprises one or more LEDs.

In some embodiments, illumination device 19 comprises two or more LEDs that each emit light in different wavelengths or at different times. In those embodiments, visualization device 18 may comprise one or more sensors capable of receiving the emitted wavelengths and may be coupled to an analytical device for reconstructing the images.

Power source 20 provides power for visualization device 18 and illumination device 19. Power source 20 as shown comprises an external source of electricity. In other embodiments, power source 20 may comprise an onboard battery. Power source 20 supplies power to, and is in communication with, visualization device 18 and illumination device 19 through conduit 21. Conduit 21 may be an insulated electrical wire or other appropriate medium for transferring energy.

Visualization device 18 is in communication with image display 22 through conduit 23. In other embodiments, visualization device 18 is in communication with image display wirelessly, such as by radio waves, infrared signals, or other known means of wireless communications. Image display 22 preferably converts the signals generated by visualization device 18 into a video image that may be displayed on a viewing screen. Image display 22 for converting the output of a CCD or CMOS chip to a video image are known in the art, and may be of the type commonly used in digital video camcorders. Image display 22 may comprise any suitable video display and may be either integral with, or separate from, power source 20.

Other features of device 10 shown in the embodiment of FIG. 1 include ventilation ports 24 and 25, used to attach an Ambu bag or other ventilation device to tracheal lumen 11 or esophageal lumen 12, respectively. Also, inflation port 26 is in communication with proximal balloon 17 through lumen 27, and inflation port 28 is in communication with distal balloon 16 through lumen 29. Balloons 16 and 17 may be selectively inflated or deflated through inflation ports 26 and 28. For example, inflation ports 26 and 28 are configured to couple with a conventional syringe such that the syringe may be used to force air into the respective balloon. In a preferred embodiment for an adult patient, distal balloon 16 may be inflated with the addition of 15 ml of air or other fluid, whereas proximal balloon 17 may be inflated with 100 ml of air or other fluid. Balloons 16 and 17 can also be deflated by coupling a syringe to the respective inflation port and retracting the plunger, as is known in the art.

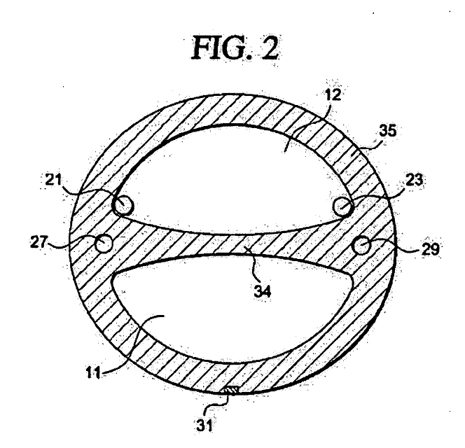

Device 10 also comprises optional markings 30. Markings 30 may comprise circumferential lines, indicia of measurements along an axial direction, or other commonly known system of indicating the proper depth of insertion of device 10. Radio-opaque marker 31 is an optional feature that also may be incorporated into device 10. In this embodiment, radio-opaque marker 31 extends along the axial length of device 10, as seen in FIG. 2. In other embodiments, other markers may be included at other locations and with other configurations, such as, without limitation, one or more radio-opaque rings encircling the device and located at one or both sides of the balloons.

As is conventional, device 10 is curved and pliable to follow the anatomical structures of a patient.

In accordance with one aspect of the present invention, device 10 is disposable and discarded after a single use. To facilitate this aspect, power connector 32 is disposed along conduit 21 to allow device 10 to be quickly coupled and uncoupled from power source 20 when using an external power supply. Likewise, signal connector 33 is disposed along conduit 23 to allow device 10 to be quickly coupled and uncoupled from image display 22. Image display 22 is a reusable unit that processes the signals from the visualization device 18 to generate images.

Referring now to FIG. 2, the cross section of device 10 taken along line 2-2 as shown in FIG. 1 is depicted. Tracheal lumen 11 and esophageal lumen 12 are separated by divider 34. Conduits 21 and 23 are shown in esophageal lumen 12, but may be located within wall 35 or any other suitable location in other embodiments. Radio-opaque marker 31 and balloon inflation lumens 27 and 29 are located within wall 35 of device 10.

The embodiment shown in FIGS. 1 and 2 takes advantage of space that is underutilized in known dual lumen airways. In this regard, in known designs of dual lumen airways, the esophageal lumen often extends to the distal end of the airway device. Nevertheless, as the ventilation through those esophageal lumens occurs from the ventilation port to the laterally-directed apertures, the space in the esophageal lumen between the apertures and the distal end remains substantially unused. The embodiment depicted in FIGS. 1 and 2 takes advantage of this space by locating a portion of visualization device 18 and/or illumination device 19 in the otherwise vacant space. In embodiments wherein the power supply is an internal battery, the battery may also reside in that space.

When positioning a portion of visualization device 18 in the distal portion of esophageal lumen 12 in device 10, circuitry and other components are preferably located in that area. It is preferable to locate as much of visualization device 18 as possible in the space at the distal portion of esophageal lumen 12 to reduce the volume of the components in the esophageal lumen and allow for a greater airflow.

Conduits 21 and 23 are relatively small compared to the cross sectional area of lumens 11 and 12, and therefore do not prevent adequate ventilation when positioned as shown in FIG. 2.

Device 10 preferably is constructed of a biocompatible clear polymer and is latex-free, although latex or other material may also be used. For adult applications, device 10 preferably has a diameter of 41 French, whereas an alternative embodiment may have a diameter of 37 French for smaller patients.

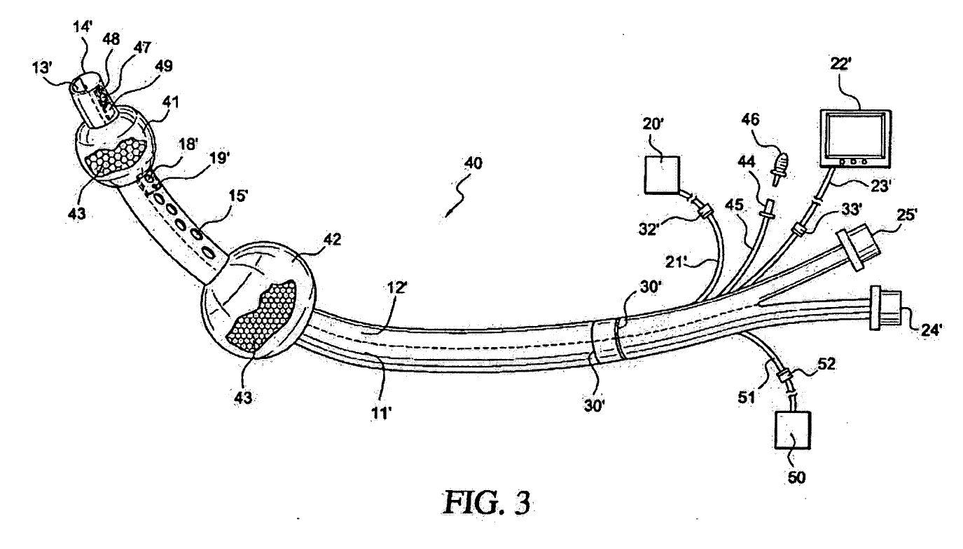

Referring now to FIG. 3, an alternative embodiment of a device in accordance with the present invention is shown. Device 40 is similar to device 10 described above and, accordingly, reference numerals having a prime (′) are similar in description as like numbered components having no prime.

One difference between device 40 and device 10 is the manner in which the apparatus is deployed. In device 10, distal balloon 16 and proximal balloon 17 are inflated by forcing air or other fluid through inflation ports 26 and 28 using a syringe. In contrast, device 40 comprises distal balloon 41 and proximal balloon 42, wherein each balloon surrounds open-cell foam 43 that may be compressed to a small volume when evacuated and that re-expands to conform to and seal the interior of a patient's trachea or esophagus when deployed. One preferred material for open-cell foam 43 is an open-cell polyurethane foam.

Balloons 41 and 42 are connected to port 44 through lumen 45. Port 44 may be obstructed with removable plug 46. When plug 46 is removed, the interior of balloons 41 and 42 are in communication with the environment. Thus, balloons 41 and 42 may be inflated from a compressed configuration by the removal of plug 46, which allows air to reach the interior of balloons 41 and 42, thereby allowing foam 43 to expand.

To deflate previously inflated balloons 41 and 42, a syringe or other suction source may be attached to port 44 to draw air or other fluid from the interior of balloons 41 and 42 and collapse those structures. This deflation may be performed prior to removal of device 40 from a patient.

Device 40 further comprises visualization device 47. Visualization device 47 is preferably disposed within esophageal lumen 12′ near distal end 14′ and distal to apertures 15′. Visualization device 47 preferably is configured to gather images from distal of device 40. Hence, this feature may assist a clinician in determining the placement of the airway as the physician may be able to visualize anatomical landmarks or features, such as rings. Additionally, the clinician may detect repositioning of device 40 by observing a change in anatomical features or landmarks as shown on display 22′.

Visualization device 47 may be used in combination with visualization device 18′ to provide different perspectives of a patient. In other embodiments, visualization device 47 and visualization device 18′ may be positioned in proximity to allow for stereoscopic vision. Visualization device 47 may communicate with display 22′ via conduit 23′, or alternatively may communicate via a second conduit or communicate with a second display.

Device 40 also comprises illumination device 48, which may be similar to illumination device 19′, and may be described in a like fashion.

Additionally, device 40 also may comprise one or more sensors 49. Sensor(s) 49 may be disposed at any convenient location and may comprise carbon dioxide sensors, microphones, nanotube field effect transistors (NTFETs), or other known sensors, and may communicate with output device 50 via conduit 51. Output device 50 may be any appropriate apparatus for communicating information obtained by sensor 49, such as a speaker, digital display, or other known apparatus. Sensor 49 may be coupled and uncoupled to output device 50 via connector 52. In other embodiments, output device 50 may be integral with device 40.

Power source 20′ may be in communication with illumination device 48, visualization device 47, and sensor 49 via conduit 21′. Alternatively, two or more power sources may be used to provide power to the components.

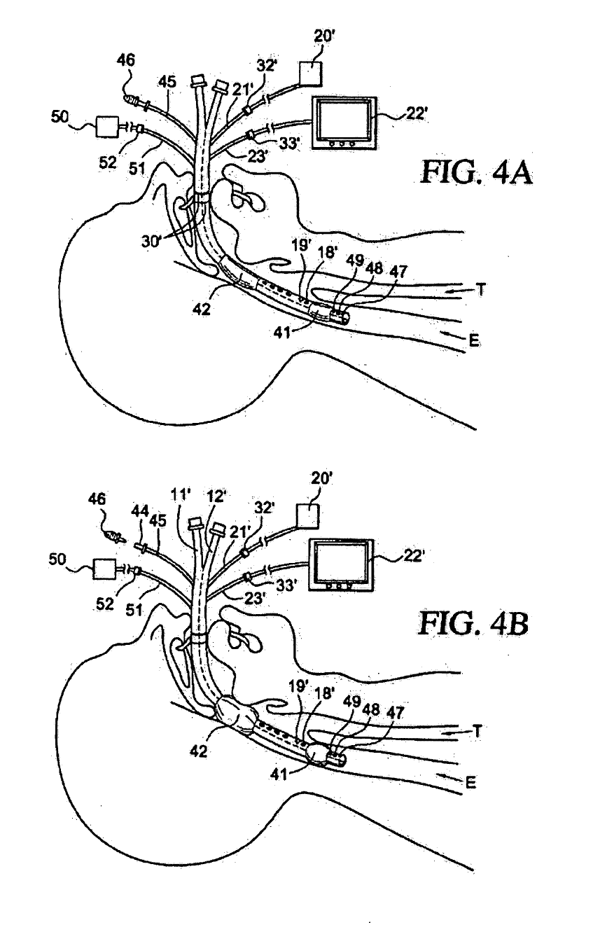

Next, a preferred method of use will be described further illustrating the benefits of an embodiment of the present invention. FIG. 4 depict several steps in a preferred method of using device 40 described above and depicted in FIG. 3.

Device 40 is preferably stored for use in a sterile container that allows rapid access to device 40. Moreover, balloons 41 and 42 are preferably stored in a collapsed configuration, such that foam 43 is compressed and device 40 has a relatively small delivery profile. Plug 46 is attached to connector 44 at proximal end of conduit 45 to prevent air from reaching the interior of balloons 41 and 42.

To prepare device 40 for use, device 40 is removed from the storage container and examined to ensure that balloons 41 and 42 have not inflated, which may indicate that plug 46 may have become dislodged. Device 40 is connected to display 22′ via connector 33′ on conduit 23′. Device 40 is connected to power supply 20′ via connector 32′ on conduit 21′. Device 40 optionally also may be connected to output device 50 via connector 52 on conduit 51.

The clinician or other individual may observe the output of visualization device 18′ and visualization device 47 on display 22′. Device 40 then may be inserted orally into a patient as the clinician observes display 22′. Device 40 may be distally advanced an appropriate distance, as may be indicated by markings 30′. The clinician may determine whether device 40 is in the patient's trachea T or esophagus E by observing anatomical features and landmarks on display 22′.

In this example, device 40 was placed into the patient's esophagus E, as depicted in FIG. 4A. At this point, the clinician may be aware of the location of device 40 by the output from visualization device 47, which does not show rings, as may be seen with placement in the trachea. Additionally, clinician may be aware of the location of device 40 based on the output of visualization device 18′, which shows the entrance to the larynx. If optional sensor 49 is used, that component may transmit additional information that may be used to determine the position of device 40.

In the event that device 40 was placed in the patient's trachea T, the clinician would have received information to indicate that placement. For example, visualization device 47 may transmit images showing rings consistent with those in the trachea T. Likewise, visualization device 18′ may transmit images that are not taken from the exterior of the entrance to the larynx. Sensor 49 may also transmit different information, such as an increased carbon dioxide reading, increased breath sounds, or other data.

Once device 40 is advanced a sufficient degree, the clinician may inflate balloons 41 and 42 by removing plug 46. After plug 46 is removed, air can travel from the environment, through conduit 45, and into the interior of balloons 41 and 42. As air reaches the interior of balloons 41 and 42, foam 43 expands, thereby inflating balloons 41 and 42 and sealing the bodily lumens in which device 40 is located. This configuration is depicted in FIG. 4B.

After device 40 is deployed by inflating balloons 41 and 42, the clinician may ascertain the position by observing display 22′ and/or output device 50.

If device 40 is positioned in the patient's esophagus E, as shown in FIG. 4B, the clinician may then ventilate the patient via esophageal lumen 12′. This ventilation may be accomplished by attaching an Ambu-bag or other source of air or oxygen to ventilation port 25′. It should be understood that if device 40 was placed in the patient's trachea T, ventilation would occur through tracheal lumen 11′. Advantageously, in either scenario, the clinician need not auscultate the patient or use a Toomey syringe to determine the position of device 40, thereby saving time and allowing oxygen to be delivered to the patient in less time than when using conventional dual lumen airway devices.

Following ventilation of the patient, and any other desired procedures, device 40 may be removed from the patient. Prior to removal, balloons 41 and 42 are preferably deflated. Port 44 preferably is adapted to be coupled to syringe S, which is a conventional syringe. Syringe S is then coupled to port 44 and the plunger is retracted to create suction and withdraw air from balloons 41 and 42 and through conduit 45. FIG. 4C depicts device 40 at a point where syringe S has been attached to port 44 and retracted to deflate balloons 41 and 42. After balloons 41 and 42 are deflated, device 40 may be withdrawn proximally from the patient, thereby completing the ventilation procedure.

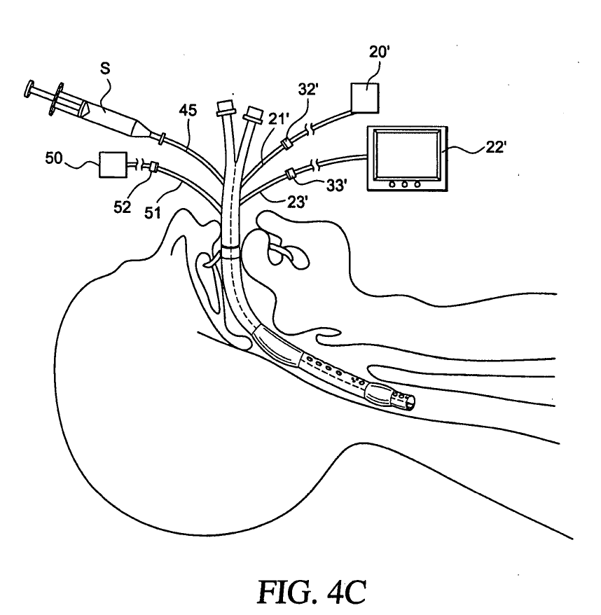

FIG. 5 depicts another preferred embodiment of the present invention that is appropriate for use in selectively providing ventilation to either of a patient's lungs during a thoracotomy. Device 60 has tracheal lumen 61 and bronchial lumen 62. Aperture 63 of bronchial lumen 62 is located at distal end 64 of device 60. Aperture 65 of tracheal lumen 61 is located between distal balloon 66 and proximal balloon 67. It will be appreciated that the device may be configured such that bronchial lumen 62 corresponds to either the left or right bronchus. As such, the device may have a nonlinear portion, such as a curve or bend, along the length between distal balloon 66 and proximal balloon 67 relative to a proximal portion of device 60.

In use, proximal balloon 67 is positioned within a patient's trachea, whereas distal balloon 66 is positioned within one of a patient's bronchi. Proximal balloon 67 is configured to obstruct the space between device 60 and the patient's trachea, and may therefore have a larger diameter than distal balloon 66, which is configured to obstruct the space between device 60 and a patient's bronchus. For example, if device was configured as a 41 French I.D., proximal balloon 67 may be configured to have a 26 mm resting diameter, whereas distal balloon 66 may be configured to have a 19 mm resting diameter. It will be appreciated by one of skill in the art that other sizes may be selected as appropriate for a number of patients, and a range of sizes may be provided, such as 28 French to 41 French. Balloons 66 and 67 optionally may comprise texture 66a and 67a, which may be configured similarly to texture 16a and 17a, respectively, as discussed in greater detail above.

Device 60 further comprises visualization device 68 located at least partially between distal balloon 66 and proximal balloon 67. In a preferred embodiment, visualization device 68 comprises a CMOS chip, and more preferably comprises a CMOS chip with analog output that may be directly interfaced with video hardware using NTSC/PAL format, as discussed above in relation to visualization device 18.

Visualization device 68 preferably is configured to reduce the delivery profile of device 60. In particular, visualization device 68 may be configured with a pixel array or other image gathering component remote from the supporting circuitry. By configuring visualization device 68 as described, the circuitry may be positioned at a desired location on device 60 or may be remotely located. The circuitry may be disposed on a circuit board, which may be rigid or flexible or otherwise, as is known in the art.

In a preferred embodiment, visualization device 68 provides analog output readable by hardware using NTSC/PAL technology. Visualization device 68 further may be reduced in size by omitting any infrared filter that would otherwise be commonly associated with a CMOS chip.

In an alternative embodiment, visualization device 68 may comprise a commercially available CMOS chip, such as a ⅓ inch CMOS chip or smaller, as is known in the art. The imaging portion of visualization device 68 preferably is embedded or potted in the wall of bronchial lumen 62 and is separated from the outside environment by an optically clear window or lens. Moreover, the focal length of visualization device 68 may be adjustable by a user or may be preselected in a manner similar to that described above in reference to visualization device 18.

Illumination device 69 is located in proximity to visualization device 68, such that illumination device 69 provides visible light, infrared light, or other illumination appropriate for visualization device 68. In the embodiment shown in FIG. 5, illumination device 69 comprises one or more LEDs located near or within distal balloon 66. In this regard, illumination may be provided to the interior and/or exterior of distal balloon 66, thereby facilitating the visual monitoring of that component.

In some embodiments, illumination device 69 comprises two or more LEDs that each emit light in different wavelengths or at different times, as described above in reference to illumination device 19.

Power source 70 provides power for visualization device 68 and illumination device 69 and is in communication with those devices 68 and 69 through conduit 71. Power source 70 and conduit 71 preferably are configured as described above in reference to power source 20 and conduit 21, respectively.

Visualization device 68 may communicate with image display 72 via conduit 73 or wirelessly, as discussed above in reference to image display 22 and conduit 23, respectively. Image display 72 preferably converts the signals generated by visualization device 68 into a video image that may be displayed on a viewing screen and may be either integral with, or separate from, the power source, also as discussed above in relation to image display 22.

Other features of device 60 shown in the embodiment of FIG. 5 include ventilation ports 74 and 75, used to attach an Ambu bag or other ventilation device to tracheal lumen 61 or bronchial lumen 62, respectively. Also, inflation port 76 is in communication with proximal balloon 67 through lumen 77, and inflation port 78 is in communication with distal balloon 66 through lumen 79. Balloons 66 and 67 may be selectively inflated or deflated through inflation ports 76 and 78, as is known in the art.

Device 60 also comprises optional markings 80 and/or optional radio-opaque marker 81, configured similarly to markings 30 and radio-opaque marker 31, discussed above. In this embodiment, radio-opaque marker 81 extends along all or part of the axial length of device 60, as seen in FIG. 5.

As is conventional, device 60 is curved and pliable to follow the anatomical structures of a patient, and may be manipulated with a stylet or other insertable device to facilitate proper placement.

In accordance with one aspect of the present invention, device 60 is disposable and discarded after a single use. To facilitate this aspect, power connector 82 is disposed along conduit 71 to allow device 60 to be quickly coupled and uncoupled from power source 70 when using an external power supply. Likewise, signal connector 83 is disposed along conduit 73 to allow device 60 to be quickly coupled and uncoupled from image display 72. Image display 72 is a reusable unit that processes signals from the visualization device 68 to generate images.

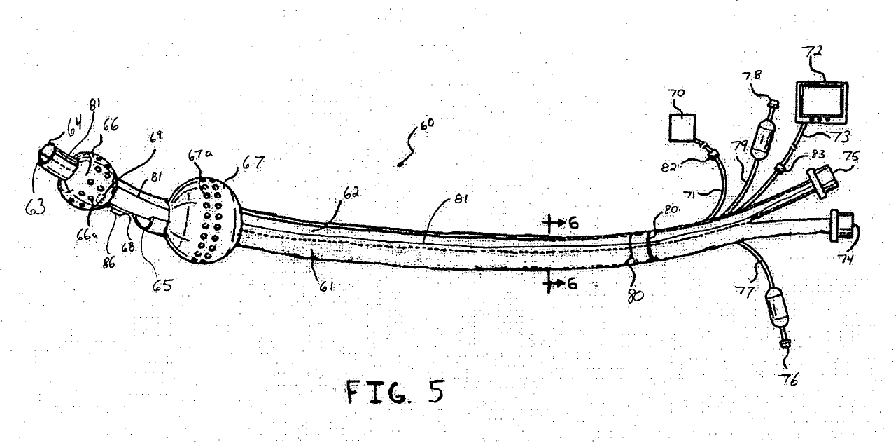

Referring now to FIG. 6, the cross section of device 60 taken along line 6-6 as shown in FIG. 5 is depicted. Tracheal lumen 61 and brachial lumen 62 are separated by divider 84. Conduits 71 and 73 are shown in tracheal lumen 62, but may be located within bronchial lumen 62, wall 85, or any other suitable location in other embodiments. Radio-opaque marker 81 and balloon inflation lumens 77 and 79 are located within wall 85 of device 60, but of course be located at any other suitable location.

Conduits 71 and 73 preferably are relatively small compared to the cross sectional area of lumens 61 and 62, and therefore do not prevent adequate ventilation when positioned as shown in FIG. 6.

Referring again to FIG. 5, another aspect of the present invention is described. Optionally, prism 86 is provided in the optical pathway of visualization device 68. Prism 86 is any device that causes light rays to deviate from a straight pathway or alter in wavelength. In this regard, prism 86 may be located atop or adjacent visualization device 68 such that light rays traveling from distal locations may be diverted onto visualization device 68. A large number of prisms suitable for medical apparatus use are per se known in the art, and may be selected for use with device 60 to obtain the desired optical pathway to visualization device 68.

Device 60 preferably is constructed of a biocompatible clear polymer and is latex-free, although latex or other material also may be used. For adult applications, device 60 preferably has a diameter of 41 French, whereas an alternative embodiment may have a diameter of 28 French for smaller patients. Device 60 is of course not limited to these sizes, and may be provided in any suitable size as desired.

Device 60 may also be configured to take advantage of other features, such as the use of sensors, additional illumination devices, and open-cell foam in balloons 66 and 67 with associated inflation and deflation mechanisms, as described above in relation to device 40 and/or FIG. 3.

Next, a preferred method of use will be described further illustrating the benefits of device 60. Device 60 preferably is stored for use in a sterile container that allows rapid access to device 60. Device 60 may be configured for placement in a left or right bronchus, but for purposes of this example will be considered to be configured to the left bronchus.

To prepare device 60 for use, device 60 is removed from the storage container and balloons 66 and 67 are each examined to ensure their integrity. Such an examination may include the inflation and deflation of each balloon. Device 60 may be attached to power supply 70 and image display 72 to ensure proper communication between device 60 and external components. Following these procedures, device 60 may then be placed in a patient.

Device 60 may be orally inserted into a patient and advanced until the distal balloon is in the left bronchus. This insertion may be performed using conventional techniques per se known in the art or may be facilitated by observation of the visual data displayed on image display 72. If using image display 72 for assistance, the progress of the advancement may be monitored and the physician may visualize the carina at the point that the trachea separates into the bronchi. Enhanced visualization may be accomplished through illumination by illumination device 69.

Balloons 66 and 67 are inflated to secure device 60 in place. Distal balloon 66 inflates to obstruct airflow through the left bronchus outside device 60, whereas proximal balloon 67 inflates to obstruct airflow through the trachea outside device 60. In this regard, airflow may still occur within device 60 via lumens 61 and 62. The physician may then observe imaging device 72 to ensure that device 60 did not become displaced upon inflation of balloons 66 and 67. In this regard, illumination device 69 provides visual or other lighting to the surrounding anatomy, which light rays may then follow a pathway through optional prism 86 (if present) and to visualization device 68. The physician may then observe a manifestation of the visualization data as displayed on the visual output of image display 72. Of course, proper placement of device 60 also may be confirmed using fluoroscopy or other conventional imaging techniques.

The physician may monitor the position of device 60 relative to anatomical landmarks in the patient, such as the carina, by observing image display 72 for changes. In particular, if a patient is undergoing a thoracotomy and must be turned, the physician may observe image display 72 for changes or any other signs that device 60 has moved or otherwise changed position.

Following the thorocotomy or other medical procedure, any devices or items placed in tracheal lumen 61 or bronchial lumen 62 may be removed, balloons 66 and 67 are deflated, and device 60 is withdrawn from the patient.

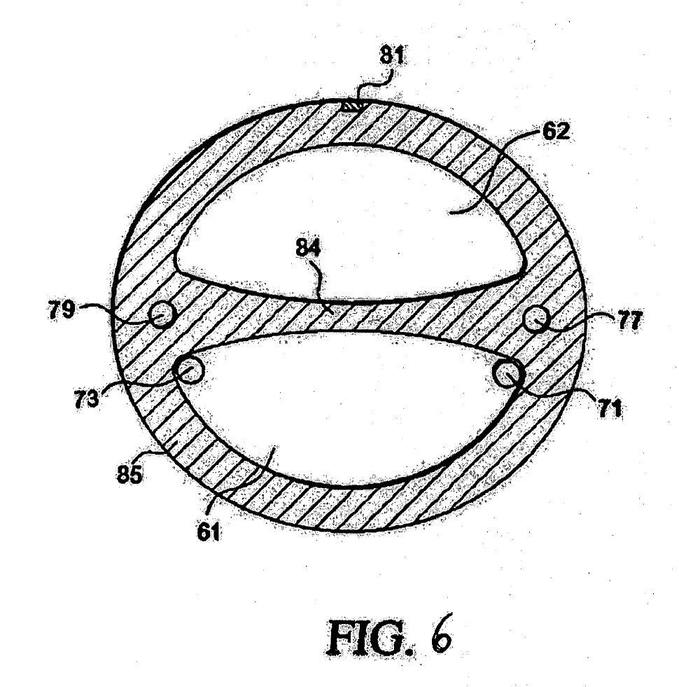

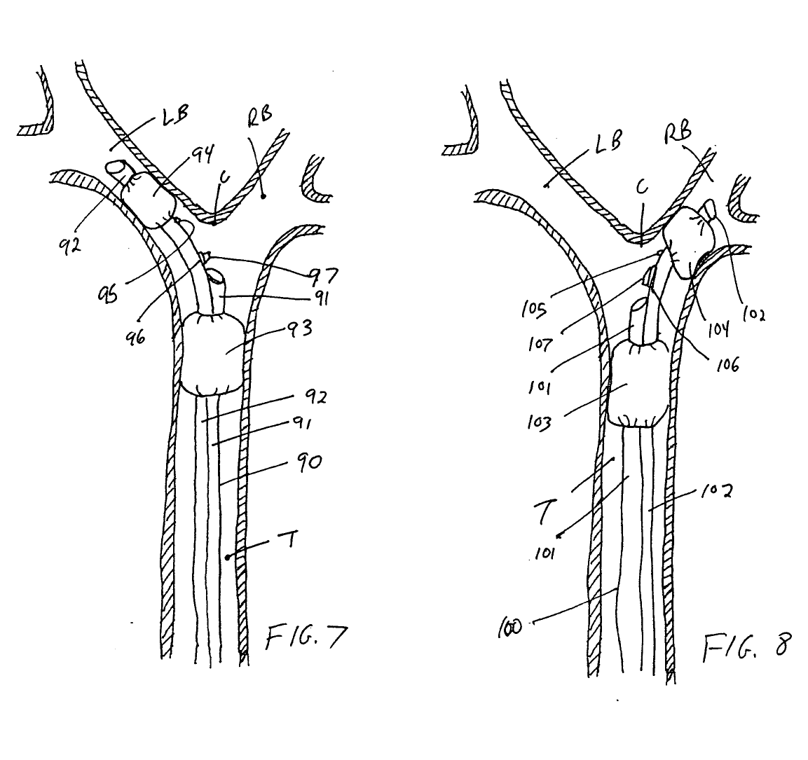

FIGS. 7 and 8 depict schematic views of two possible embodiments of an airway in accordance with the present invention. In FIG. 7, device 90 is configured to obstruct a patient's left bronchus. Device 90 comprises tracheal lumen 91, bronchial lumen 92, proximal balloon 93, distal balloon 94, illumination device 95, visualization device 96, and prism 97. Device 90 is inserted into trachea T such that distal balloon 94 is advanced beyond carina C and into left bronchus LB. This placement is facilitated by curvature of device 90 between distal balloon 94 and proximal balloon 93. Illumination device 95, visualization device 96 and prism 97 are located on a lateral aspect of bronchial lumen 92 to facilitate visualization of carina C and the opening of the right bronchus RB. One of skill in the art will appreciate that prism 97 may be positioned to direct light from the appropriate anatomical landmarks to visualization device 96.

FIG. 8 depicts device 100 that is similar in construction and operation as device 90, but is configured to obstruct the right bronchus RB. Device 100 comprises tracheal lumen 101, bronchial lumen 102, proximal balloon 103, distal balloon 104, illumination device 105, visualization device 106, and prism 107. Device 100 is inserted into trachea T such that distal balloon 104 is advanced beyond carina C and into right bronchus RB. This placement is facilitated by curvature of device 100 between distal balloon 104 and proximal balloon 103. Illumination device 105, visualization device 106 and prism 107 are located on a lateral aspect of bronchial lumen 102 to facilitate visualization of carina C and the opening of the left bronchus LB. One of skill in the art will appreciate that prism 106 may be positioned to direct light from the appropriate anatomical landmarks to visualization device 105.

It will be understood by one of skill in the art that other features of devices 90 and 100 may also be present in the proximal portions of those devices, although not specifically shown in FIGS. 7 and 8. For example, devices 90 and 100 also will be understood to comprise ventilation ports and other features similar to those disclosed above.

It is believed that the operation and construction of the present invention will be apparent from the foregoing description and, while the invention shown and described herein has been characterized as particular embodiments, changes and modifications may be made therein without departing from the spirit and scope of the invention as defined in the following claims.

What is claimed is:

1. An airway device comprising:
an elongated tube having a proximal end, a distal end, and a first lumen extending therebetween;
a first balloon circumferentially disposed on the tube near the distal end;
a second balloon circumferentially disposed on the tube proximal of the first balloon;
a second lumen extending at least partially between the proximal end and distal end and having an opening between the first and second balloons; and
a visualization device disposed within the tube,
wherein the tube is has a non-linear portion between the first and second balloons.

2. The device of claim 1 further comprising an illumination device disposed within the tube.

3. The device of claim 2 further comprising a prism in the optical pathway of the visualization device.

4. The device of claim 3 wherein the visualization device is disposed at least partially within the first lumen.

5. The device of claim 4 wherein the illumination device is disposed at least partially within the first lumen.

6. The device of claim 4 wherein the illumination device is disposed at least partially within the area bounded by the first balloon.

7. The device of claim 5 wherein the illumination device comprises a first LED that is configured to emit a first wavelength of light and a second LED that is configured to emit a second wavelengths of light.

8. The device of claim 1 further comprising one or more markings on the tube.

9. The device of claim 8 further comprising a radio-opaque marker disposed along at least a portion of the length of the tube.

10. The device of claim 9 further comprising texture on at least one of the balloons.

11. An airway device comprising:
an elongated tube having a proximal end, a distal end, and a first lumen extending therebetween;
a first balloon circumferentially disposed on the tube near the distal end;
a second balloon circumferentially disposed on the tube proximal of the first balloon;
a second lumen having an opening between the proximal end and distal end;
a visualization device attached to the tube; and
a prism in the optical pathway of the visualization device.

12. The device of claim 11 further comprising an illumination device disposed within the tube.

13. The device of claim 12 wherein the visualization device is disposed between the first and second balloons.

14. The device of claim 13 wherein the illumination device is disposed at a location distal to the visualization device.

15. The device of claim 14 wherein the tube has a non-linear portion between the first and second balloon.

16. The device of claim 15 wherein the illumination device comprises two or more LEDs.

17. The device of claim 16 further comprising one or more markings on the tube.

18. The device of claim 17 further comprising a radio-opaque marker disposed along at least a portion of the length of the tube.

19. The device of claim 18 further comprising texture on at least one of the balloons.

20. A method of ventilating a patient comprising:
providing an airway device comprising a first lumen, a second lumen, a visualization device, a prism, a first balloon, and a second balloon;
inserting the airway device into an anatomical passage of a patient;
expanding the first balloon in the anatomical passage;
expanding the second balloon in a branch of the anatomical passage;
receiving data communicated from the visualization device;
determining the position of the airway device based on the data communicated from the visualization device; and
ventilating to the patient through at least one of the lumens.

21. The method of claim 20 further comprising monitoring the data communicated from the visualization device for changes.
